(19) United States
(12) Patent Application Publication
(10) Pub. No.: US 2002/0192731 A1
(43) Pub. Date: Dec. 19, 2002

(54) Method and composition for sterilizing surgical instruments

(76) Inventor: Jason C. H. Shih, Cary, NC (US)

Correspondence Address: Steven J. Hultquist, Intellectual Property/Technology Law, P.O. Box 14329, Research Triangle Park, NC, 27709, US

(21) Appl. No.: 10/007,613

(22) Filed: Oct. 26, 2001

Related U.S. Application Data
(63) Continuation-in-part of application No. 09/834,284, filed on Apr. 12, 2001.

(51) Int. Cl. G01N 33/53; G01N 33/537; G01N 33/543; C11D 1/00; G01N 33/569; C12Q 1/02; C12Q 1/22; C12Q 1/18; C12Q 1/04; C12N 15/09; C07F 1/00; C07K 1/00; C07H 1/00; C07J 1/00; C07C 1/00; C07D 201/00

(52) U.S. Cl. 435/7.92; 510/161; 435/262; 435/31; 435/32; 435/29; 435/7.22; 435/69.2; 435/34

(57) ABSTRACT

A method and composition for sterilizing articles that are contaminated with infectious prion protein, such as surgical instruments, kitchen utensils, laboratory tools, etc., comprising the steps of: (a) heating the articles to be treated at a moderate temperature well below the incineration temperature of said infectious prion protein, wherein said moderate temperature is sufficient to enhance the proteolytic susceptibility of infective prion protein associated with said articles; and (b) exposing the heated articles to a proteolytic enzyme that is effective for at least partial reduction of the infective protein prion associated with said articles under said moderate temperature.

### CROSS-REFERENCE TO RELATED APPLICATION

This is a continuation-in-part of pending U.S. patent application Ser. No. 09/834,284 filed Apr. 12, 2001.

### FIELD OF THE INVENTION

The present invention generally relates to a composition and method for disinfecting and sterilizing medical devices and like articles that may be contaminated by infectious prion proteins associated with transmissible spongiform encephalopathy (TSE), e.g., Creutzfeldt-Jakob disease (CJD), bovine spongiform encephalopathy (BSE), and sheep scrapie. More specifically, the invention relates to use of proteases for cleansing surgical instruments and reducing or preventing contamination of such surgical instruments by infectious prion proteins.

### BACKGROUND OF THE INVENTION

Prion proteins are conformationally anomalous proteins that are associated with infectious neurodegenerative diseases in human as well as non-human mammalian species.

Prion diseases in non-human mammalian species include scrapie (sheep), transmissible mink encepohalopathy (minks), chronic wasting disease (elk, deer), bovine spongiform encephalopathy (BSE) (cows), feline spongiform encephalopathy (cats), and simian spongiform encephalopathy (monkeys).

In humans a variety of neurodegenerative conditions are etiologically associated with prion proteins, including Creutzfeldt-Jakob disease, Gerstmann-Sträussler-Scheinker syndrome, Fatal Familial Insomnia, kuru, and new variant Creutzfeldt-Jakob disease. Pathogenesis of human prion diseases is associated with carnivorism (BSE-infected beef, causing new variant Creutzfeldt-Jakob disease), administration of human growth hormone (causing iatrogenic Creutzfeldt-Jakob disease) and ritualistic cannibalism (causing kuru).

Over 180,000 BSE cases and over 100 human Creutzfeldt-Jakob disease cases have been reported in Europe since 1992, and the human cases are predicted to significantly rise. The spread of such disease is difficult to contain, since such disease has no cure and the pathogenic prion protein is recalcitrant and non-immunogenic. The pathogenic and infectious isoform of prion protein is very stable, rich in β-sheet structure, and resistant to heat and common proteolytic enzymes (Prusiner, S. B., Proc. Natl. Acad. Sci. U.S.A., 95, 11363 (1998); Cohen, F. E. and Prusiner, S. B., Ann. Rev. Biochem., 67, 793 (1998); and Pan, K-M, Baldwin, M., Nguyen, J., Gasset, M., Serban, A., Groth, D., Mehlhorn, I., Huang, Z., Fletterick, R. J., Cohen, F. E., and Prusiner, S. B., Proc. Natl. Acad. Sci. U.S.A, 90, 10962 (1993)).

Significant efforts have been focused on studies on the control of BSE and prion protein of propagation in both human and bovine species.

The cross-contamination caused by reuse of medical instruments that have been previously exposed to prion-infected tissues is becoming an increased hazard and potential contributor to the transmission of infection.

The use of antiseptics, disinfectants, and sterilization procedures in health care facilities is critical to prevent the cross-contamination by medical instruments used during health care procedures. Disinfection and sterilization of medical devices or instruments are achieved by a variety of conventional methods, using various physical and chemical processes that destroy infectious biological materials, such as bacteria or viruses. For example, chemical disinfectants such as peracetic acid, hydrogen peroxide, sodium hydroxide, formic acid, bleach, alcohols, ethylene oxide, formaldehyde, formalin, and glutaraldehyde can be used for disinfecting and sterilizing medical devices; incineration, autoclaving, freezing, dry heating, boiling, UV and microwave radiation are also useful for destructing traditional infectious agents such as bacteria and viruses.

However, these conventional methods are ineffective for disinfecting or sterilizing prion-contaminated medical devices or similar articles, due to the fact that infectious prion proteins are resistant to destruction by conventional methods that denature and otherwise degrade conformationally normal proteins.

It therefore would be a significant advance in the art to provide a composition and methodology for effectively disinfecting or sterilizing prion-infected medical devices such as surgical instruments, or like articles such as kitchen utensils and laboratory tools.

### SUMMARY OF THE INVENTION

The invention provides a method and composition for disinfecting or sterilizing medical devices such as surgical instruments that are contaminated by infectious prion proteins.

In one aspect, the invention relates to a disinfection method for cleansing prion-contaminated surgical instruments, comprising the steps of:

(a) heating the contaminated surgical instruments to a sufficient temperature and for sufficient time to enhance the proteolytic degradability of infective prion protein associated with said instruments; and

(b) exposing the heated instruments to a proteolytic enzyme that is effective for at least partial reduction of infective prion protein associated with said prion-contaminated surgical instruments.

The method of the present invention have proved to be particularly effective for disinfecting or sterilizing surgical instruments, including but not limited to: clamps, forceps, scissors, knives, cables, punches, tweezers, cannulae, calipers, carvers, curettes, scalers, dilators, clip applicators, retractors, contractors, excavators, needle holders, suction tubes, trocars, coagulation electrodes, electroencephalographic depth electrodes, rib and sternum spreaders, bipolar probes, rib shears, etc.

In another aspect of the present invention, the above-described method is used for treating/cleansing cutleries and kitchen utensils that are susceptible to contamination by prion-infected bovine tissues, such as knives, forks, scissors, peelers, parers, slicers, spatulas, cleavers, etc.

In yet another aspect of the present invention, the above-described method is employed for disinfecting or sterilizing veterinary tools and devices, such as clamps, forceps, knives, saws, probes, and electronic stun equipment.

In a still further aspect of the present invention, the above-described method is useful for sterilizing laboratory tools that have been used for isolating, purifying, and characterizing prion proteins. Such laboratory tools are those generally used in gel filtration, ion exchange, HPLC chromatography, electrophoresis, western blotting, isoelectric focusing, etc., including but not limited to filtration devices, centrifuges, spectrophotometers, fluorometers, and various containers.

The treatment temperature of the present invention generally does not exceed about 150° C., and preferably is at least 35° C. More preferably, the treatment temperature in step (a) of the present method is within the range of from about 100° C. to about 150° C., and most preferably within the range of from about 125° C. to about 140° C.

It is preferred that step (b) of the present method is carried out at a temperature that is lower than that of the step (a), for instance, within the range of from about 35° C. to about 100° C. More preferably, the temperature in step (b) is within the range of from about 35° C. to about 75° C., and most preferably within the range of from about 50° C. to about 65° C. at a pH range of from about 6.0 to about 9.5.

The proteolytic enzyme useful for the present invention include, but are not limited to, keratinase enzymes, proteinase K, trypsins, chymotrypsins, pepsins, chymosins, cathepsins, subtilisins, elastases, collagenases, endopeptidases, peptidases, oligopeptidease, thermolysins, bacillolysin, mycilysins, carboxypeptidases, leucyl aminopeptidases, aminopeptidases, extremthermophilic proteases, carbonyl hydrolase, papain, pancreatin, streptokinase, streptokinase, ficin, carboxypeptidase, chymopapain, and bromelin.

Keratinase enzymes are particularly preferred for the practice of the present invention. Keratinases are a group of proteolytic enzymes that are generally known as being capable of breaking down keratin proteins that are the major components of feather, horn, hooves, and hair. The inventor of the present application has discovered an unexpected and surprising result that Keratinase enzymes are also effective in destructing infectious prion proteins, especially if the infectious prion proteins have been rendered proteolyticly susceptible.

Another preferred proteolytic enzyme is a mutant of wild-type Bacillus amyloliquefaciens subtilisin, comprising one or more amino acid substitutions, additions, or deletions.

The proteolytic enzyme of the present invention is preferably provided in a solution form for purpose of cleansing and sterilizing the surgical instruments or like articles. While keratinases are used as the proteolytic enzyme, such enzyme solution is preferably characterized by a low effective concentration within the range of from about 0.2 g/L to about 1.0 g/L.

Another aspect of the invention relates to a method of removing infectious prion protein from surgical instruments by proteolytic enzymatic degradation treatment, including (a) heating the surgical instruments at a temperature that is below the pyrolytic destruction temperature of the prion protein (i.e., the temperature (>>200° C.) that is normally employed for incineration of the prion protein) but is sufficient to render said infectious prion protein susceptible to enzymatic degradation, followed by (b) enzymatic degradation treatment of the prion protein.

A still further aspect of the present invention comprises the steps of heating prion-infected articles at a temperature in a range of from about 100° C. to about 150° C., e.g., for a time of from about 5 minutes to about 5 hours, followed by (b) exposing the heated articles to a proteolytic enzyme at a temperature in a range of from about 35° C. to about 75° C. at which the proteolytic enzyme is thermally stable and proteolytically effective to at least partially destroy the infective prion protein associated with the bovine tissue.

A still further aspect of the invention relates to a method of at least partially degrading infectious prion protein in surgical instruments contaminated with same, by steps including heating the surgical instruments and simultaneously exposing same to a thermal stable proteolytic enzyme, at sufficient temperature and for sufficient time to at least partially degrade the infectious prion protein associated with such surgical instruments.

In one compositional aspect, the invention relates to a cleansing composition for sterilizing surgical instruments and like articles, comprising (i) one or more proteolytic enzyme(s) that are thermally stable in a temperature range of from about 35° C. to about 100° C.; (2) a solvent. The proteolytic enzyme useful for the present invention include, but are not limited to, keratinase enzymes, proteinase K, trypsins, chymotrypsins, pepsins, chymosins, cathepsins, subtilisins, elastases, collagenases, endopeptidases, peptidases, oligopeptidease, thermolysins, bacillolysin, mycilysins, carboxypeptidases, leucyl aminopeptidases, aminopeptidases, extremthermophilic proteases, carbonyl hydrolase, papain, pancreatin, streptokinase, streptodornase, ficin, carboxypeptidase, chymopapain, and bromelin. Preferably, the cleansing composition of the present invention comprises Keratinase enzymes in the concentration range of from about 0.2 g/L to 1.0 g/L. Various solvents can be used for the purpose of practicing the present invention, such as distilled water, buffer solution, detergent solution, alcohol, or any other inorganic or organic solvent commonly used in enzymatic detergents, which can be readily determined by a person ordinarily skilled in the art without undue experimentation. Preferably, the cleansing composition further comprises one or more chemical additives for enhancing the disinfection/sterilization results, which include but are not limited to: surfactants, builders, boosters, fillers, and other auxiliaries.

Other aspects, features and embodiments of the invention will be more fully apparent from the ensuing disclosure and appended claims.

### DETAILED DESCRIPTION AND PREFERRED EMBODIMENTS OF THE INVENTION

Relative to the present invention and its features, aspects and embodiments as more fully described hereinafter, the disclosures of the technical literature cited in the Background of the Invention section hereof, as well as the following patents and technical literature articles, are hereby incorporated herein by reference in their respective entireties:

U.S. Pat. Nos.: 4,760,025, 4,908,220; 4,959,311; 5,063,161; 5,171,682; 5,186,961; and 5,712,147;

Deslys, J. P., “Screening slaughtered cattle for BSE,” Nature, Vol. 409, pp. 476-477, Jan. 25, 2001; and

Cohen, F. E., “Protein Misfolding and Prion Diseases,” J. Mol. Biol. (1999), Vol. 293, pp. 313-320.

The present invention is based on the use of proteolytic enzymes for disinfection or sterilization of prion-contaminated articles such as surgical instruments, cutlery and kitchen utensils, veterinary tools, and laboratory tools.

The efficacy of the process of the present invention for degradation of the infectious prion protein is wholly unexpected since high temperature exposure (e.g., at 200° C.) of infectious prion proteins alone does not alter their pathogenic character; additionally, conventional proteolytic enzymes such as proteinase K that fully digest non-infectious PrP^{c} do not destroy the corresponding infectious isoform. It therefore is highly surprising that temperatures well below the incineration temperatures heretofore necessary for destruction of infectious PrP^{Sc} can be employed for enzymatic treatment, to totally eliminate infectious PrP^{Sc} from tissue containing or contaminated with same.

As used herein, the term elevated temperature means temperature of at least 35° C. The term proteolytic susceptibility means the ability of an infective prion protein to be enzymatically degraded to a non-infective product.

The disinfection/sterilization of surgical instruments can be carried out by various techniques as hereinafter described.

For example, in one embodiment of the invention, the surgical instruments to be treated is heated to a sufficient temperature and for sufficient time to enhance the proteolytic susceptibility of infective prion protein that may be present, in conjunction with exposure of the tissue to a proteolytic enzyme that is effective to at least partially destroy any infective prion protein that is present.

The disinfection/sterilization process can also be carried out in a two-step sequence, including an initial step of heating the surgical instruments to a first higher elevated temperature and then exposing the heated instruments to the enzymatic agent at a second lower elevated temperature, for proteolytic degradation of the infectious prion protein associated with such surgical instruments.

The method can therefore be carried out in various embodiments in which proteolytic susceptibility of infective prion protein in the surgical instruments is enhanced by heating of the tissue to an elevated temperature for subsequent proteolytic enzyme treatment. The elevated temperature in the heating step may be any suitable temperature, e.g., at least 35° C., at least 40° C., at least 60° C., at least 75° C., and/or no more than 150° C. (or other lower temperature, as desired), with one illustrative specific temperature range being from about 100° C. to about 150° C., and more preferably from about 125° C. to about 140° C.

Alternatively, the disinfection/sterilization process of the invention may be carried out in a single-step procedure, in instances where the proteolytic enzyme is stable and effective (to remove the infectious prion protein) at the corresponding temperature used in the treatment process, so that no initial heating step is required.

In the single step method, the prion-contaminated surgical instruments or like articles are heated to a suitable elevated temperature for the enzymatic degradation of the infectious prion protein to occur.

For example, the method of at least partially degrading infectious prion protein in prion-contaminated surgical instruments or like articles can be carried out by heating and simultaneously exposing the surgical instruments or like articles to a thermal stable proteolytic enzyme, at sufficient temperature and for sufficient time to at least partially degrade the infectious prion protein.

Temperature in the enzymatic treatment may be widely varied in the broad practice of the invention, depending on the thermostable character of the proteolytic enzyme that is used to degrade the infectious prion protein.

It will be recognized that any of a wide variety of proteases may be employed in the practice of the invention, and that the choice of specific proteolytic enzyme will affect the choice of temperature that is used to carry out the proteolytic degradation, as well as the choice of any elevated temperature treatment of the tissue before its exposure to the proteolytic enzyme.

Specific temperature treatment conditions for the enzymatic treatment, as well as the temperature conditions necessary or desirable for any elevated temperature initial treatment step(s) that precede such enzymatic treatment, can be readily empirically determined without undue experimentation, within the skill of the art.

Proteolytic enzymes usefully employed in the practice of the invention include enzymes that are enzymatically active and effective at the conditions of their use. For elevated temperature enzymatic treatment, the proteolytic enzyme is suitably thermostable at the conditions of use.

In this respect, proteolytic enzymes of widely varying thermostable character are known. For example, various proteolytic enzymes employed in specific embodiments of the invention may be thermostable up to 35° C., 40° C., 50° C., 60° C. or even 100° C.

The proteolytic enzyme may be of any suitable type, and may comprise a single enzymatic species, or alternatively a mixture of enzymes. The enzyme may be used in a purified and concentrated form, or alternatively in a diluted form. It is preferred that the enzyme is dissolved in a solvent to form an enzyme solution with a concentration of from about 0.2 g/L to about 1.0 g/L.

Illustrative proteolytic enzymes in the broad practice of the present invention include, without limitation, keratinase enzymes, proteinase K, trypsins, chymotrypsins, pepsins, chymosins, cathepsins, subtilisins, elastases, collagenases, endopeptidases, peptidases, oligopeptidease, thermolysins, bacillolysin, mycilysins, carboxypeptidases, leucyl aminopeptidases, aminopeptidases, extremthermophilic proteases, carbonyl hydrolase, papain, pancreatin, streptokinase, streptodornase, ficin, carboxypeptidase, chymopapain, and bromelin.

Preferred enzyme species include keratinase enzymes. A particularly preferred keratinase comprises Bacillus licheniformis PWD-1 keratinase. Proteolytic enzyme species useful in the practice of the invention include active fragments of proteolytic enzymes, e.g., an active fragment of a keratinase enzyme, such as the Bacillus licheniformis PWD-1 keratinase. When keratinase enzymes are used in the present invention, the effective concentration required for the enzyme solution is significantly lower than that of the conventional enzyme detergents. Moreover, keratinase enzymes are characterized by an optimal active temperature range of from about 50° C. to about 65° C. at pH value of from about 6.0 to about 9.5, which is significantly higher than that of most conventional enzyme detergents. Therefore, the cleansing temperature of the process of the present invention can be significantly increased, which is more efficient for enhancing the proteolytic susceptibility of the infective prion protein associated with the surgical instruments.

The method of enzymatically removing infectious prion proteins from tissue, in accordance with the present invention, can further include the step(s) of testing the tissue to verify destruction of infective prion protein therein, after proteolytic enzymatic treatment has been concluded.

The disinfection/sterilization method of the invention can be carried out in any suitable manner, with any appropriate sequence of processing steps.

For example, in one embodiment, the surgical instruments to be treated are subjected to initial non-enzymatic thermal treatment as necessary or desired, followed by enzymatic treatment for destruction of infective or contaminative prion protein, followed by rinsing and non-enzymatic treatment, and further thermal/enzymatic treatment (e.g., in an alternating and repetitive cycle of non-enzymatic thermal treatment, and enzymatic elevated temperature treatment).

The following table shows a disinfection/sterilization cycle according to one embodiment of the present invention:

TABLE I

| Steps | Temperature | Time | |
|---|---|---|---|
| Pre-Wash | Room Temp. | 2-5 | minutes |
| (cold water) | | | |
| Heating | 35-100° C. | 20-40 | minutes |
| Cooling | 34-51° C. | 2-10 | minutes |
| Enzyme Wash | 34-51° C. | 20-120 | minutes |
| Sonication | 34-51° C. | 5 | minutes |
| Detergent Wash | 51-57° C. | 2-5 | minutes |
| Rinse and Dry | Room Temp. | 5 | minutes |

| | | |
|---|---|---|
| Autoclave Sterilization | 200-500° C. | — |

In another embodiment, a thermally stable proteolytic enzyme is used, so that the heating and enzymatic wash steps can be conducted simultaneously, at sufficient temperature and for sufficient time for complete destruction of the infection prion protein and sterilization of the surgical instruments.

The method of the invention is broadly applicable to the destruction of prion protein contaminates associated with:

(1) surgical instruments, such as clamps, forceps, scissors, knives, cables, punches, tweezers, cannulae, calipers, carvers, curettes, scalers, dilators, clip applicators, retractors, contractors, excavators, needle holders, suction tubes, trocars, coagulation electrodes, electroencephalographic depth electrodes, rib and sternum spreaders, bipolar probes, rib shears, etc.;

(2) cutlery and kitchen utensils, such as knives, forks, scissors, peelers, parers, slicers, spatulas, and cleavers; and

(3) laboratory tools, such as filtration devices, centrifuges, spectrophotometers, fluorometers, and various containers and

(4) veterinary tools and devices, such as clamps, forceps, knives, saws, probes and electronic stun equipment.

The above list is only illustrative of several application of the present invention, and it should not be construed in any manner as to limit the scope of the present invention.

The present invention in one compositional aspect comprehends a cleansing composition including (i) a proteolytic enzyme, e.g., Bacillus licheniformis PWD-1 keratinase, that is thermally stable in the temperature range employed for enzymatic treatment, e.g., from about 40° C. to about 60° C.; and (ii) a solvent.

Any solvent that is suitable for use with enzymatic detergents can be employed in the composition of the present invention. Distilled water is a preferred solvent, in light of its biological compatibility and low costs. Other conventional inorganic and organic solvents, such as alcohol, buffer solution, detergent solution, can also be used for purpose of practicing the present invention, and a person ordinarily skilled in the art can readily select solvents that are compatible with specific enzymes used. Chemical additives that are conventionally employed can also be introduced into the cleansing composition of the present invention, which include but are not limited to surfactants, builders, boosters, fillers, and other auxiliaries.

The cleansing composition of the present invention retains its effectiveness for destructing infectious prion protein even at a very low concentration, for example, of less than 0.3 g/L. When keratinase enzyme is employed in such cleansing composition, the enzyme concentration of such composition is preferably within the range of from about 0.2 g/L to about 1.0 g/L.

The cleansing composition of the present invention is enzymatically reactive at elevated temperature, and it therefore may be used at elevated temperature for complete destruction of infectious prion protein associated with surgical instruments, cutleries, kitchen utensils, veterinary tools, and laboratory tools.

While the invention has been described herein with reference to various illustrative features, aspects, and embodiments, it will be appreciated that the utility of the invention is not thus limited, but rather extends to and encompasses other variations, modifications and other embodiments, as will readily suggest themselves to those of ordinary skill in the art.

Accordingly, the invention is to be broadly interpreted and construed as including such other variations, modifications and other embodiments, within the spririt and scope of the invention as hereinafter claimed.

1. A method of disinfecting article(s) that are susceptible to contamination by infectious prion protein, the method comprising the steps of:
(a) heating said article(s) to a sufficient temperature and for sufficient time to enhance the proteolytic susceptibility of infective prion protein associated with said article(s); and
(b) exposing the heated article(s) to a proteolytic enzyme that is effective for at least partial reduction of the infective protein prion associated with said article(s).

2. The method of claim 1, wherein said articles comprise surgical instruments.

3. The method of claim 2, wherein said surgical instrument(s) are selected from the group consisting of: clamps, forceps, scissors, knives, cables, punches, tweezers, cannulae, calipers, carvers, curettes, scalers, dilators, clip applicators, retractors, contractors, excavators, needle holders, suction tubes, trocars, coagulation electrodes, electroencephalographic depth electrodes, rib and sternum spreaders, bipolar probes, and rib shears.

4. The method of claim 1, wherein said article(s) comprise cutleries and kitchen utensils.

5. The method of claim 4, wherein said cutleries and kitchen utensils are selected from the group consisting of: knives, forks, scissors, peelers, parers, slicers, spatulas, and cleavers.

6. The method of claim 1, wherein said article(s) comprise laboratory apparatus(es).

7. The method of claim 6, wherein said laboratory apparatus(es) are selected from the group consisting of: containers, filtration devices, centrifuges, spectrophotometers, and fluorometers.

8. The method of claim 1, wherein said article(s) comprise veterinary devices.

9. The method of claim 8, wherein said veterinary devices are selected from the group consisting of clamps, forceps, knives, saws, probes, and electronic stun equipment.

10. The method of claim 1, wherein the temperature in step (a) comprises a temperature not exceeding about 150° C.

11. The method of claim 1, wherein the temperature in step (a) comprises a temperature of at least 35° C.

12. The method of claim 1, wherein the temperature in step (a) comprises a temperature below about 150° C.

13. The method of claim 1, wherein the temperature in step (a) comprises a temperature in a range of from about 100° C. to about 150° C.

14. The method of claim 1, wherein the temperature in step (a) comprises a temperature in a range of from about 125° C. to about 140° C.

15. The method of claim 1, wherein step (b) is conducted at lower temperature than step (a).

16. The method of claim 1, wherein step (b) is carried out at temperature above about 40° C.

17. The method of claim 1, wherein step (b) is carried out at temperature above about 50° C.

18. The method of claim 1, wherein step (b) is carried out at temperature in a range of from about 35° C. to about 75° C.

19. The method of claim 1, wherein step (b) is carried out at temperature in a range of from about 40° C. to about 75° C.

20. The method of claim 1, wherein step (b) is carried out at temperature in a range of from about 50° C. to about 65° C.

21. The method of claim 1, wherein the proteolytic enzyme comprises at least one enzyme selected from the group consisting of keratinase enzymes, proteinase K, trypsins, chymotrypsins, pepsins, chymosins, cathepsins, subtilisins, elastases, collagenases, endopeptidases, peptidases, oligopeptidease, thermolysins, bacillolysin, mycilysins, carboxypeptidases, leucyl aminopeptidases, aminopeptidases, extremthermophilic proteases, carbonyl hydrolase, papain, pancreatin, streptokinase, streptodornase, ficin, carboxypeptidase, chymopapain, and bromelin.

22. The method of claim 1, wherein the proteolytic enzyme comprises a keratinase enzyme.

23. The method of claim 1, wherein the proteolytic enzyme comprises an active fragment of a keratinase enzyme.

24. The method of claim 1, wherein the proteolytic enzyme comprises a Bacillus licheniformis PWD-1 enzyme or an active fragment thereof.

25. The method of claim 1, wherein the proteolytic enzyme comprises a protease enzyme.

26. The method of claim 25, wherein the protease enzyme comprises a carbonyl hydrolase.

27. The method of claim 26, wherein the carbonyl hydrolase comprises subtilisin.

28. The method of claim 27, wherein the subtilisin comprises a mutant of wild-type Bacillus amyloliquefaciens subtilisin, comprising one or more amino acid substitutions, additions, or deletions.

29. The method of claim 25, wherein the protease enzyme comprises at least one enzyme selected from the group consisting of: papain, pancreatin, trypsin, chymotrypsin, pepsin, streptokinase, streptodornase, ficin, carboxypeptidase, aminopeptidase, chymopapain, bromelin, and subtilisin.

30. A method of removing infective prion protein from a surgical instrument contaminated with same, the method including (a) heating the surgical instrument at a temperature in a range of from about 100° C. to about 150° C., followed by (b) exposing the heated surgical instrument to a proteolytic enzyme at a temperature in a range of from about 35° C. to about 100° C. at which the proteolytic enzyme is thermally stable and proteolytically effective to at least partially destroy the infective prion protein contaminating said surgical instrument.

31. The method of claim 30, wherein said heating is conducted for a time of from about 5 minutes to about 5 hours.

32. The method of claim 30, wherein the proteolytic enzyme comprises at least one enzyme selected from the group consisting of keratinase enzymes, proteinase K, trypsins, chymotrypsins, pepsins, chymosins, cathepsins, subtilisins, elastases, collagenases, endopeptidases, peptidases, oligopeptidease, thermolysins, bacillolysin, mycilysins, carboxypeptidases, leucyl aminopeptidases, aminopeptidases, extremthermophilic proteases, carbonyl hydrolase, papain, pancreatin, streptokinase, streptodornase, ficin, carboxypeptidase, chymopapain, and bromelin.

33. The method of claim 30, wherein the proteolytic enzyme comprises Bacillus licheniformis PWD-1 keratinase.

34. The method of claim 1, wherein the proteolytic enzyme comprises a protease enzyme.

35. The method of claim 34, wherein the protease enzyme comprises a carbonyl hydrolase.

36. The method of claim 35, wherein the carbonyl hydrolase comprises subtilisin.

37. The method of claim 36, wherein the subtilisin comprises a mutant of wild-type Bacillus amyloliquefaciens subtilisin, comprising one or more amino acid substitutions, additions, or deletions.

38. The method of claim 34, wherein the protease enzyme comprises at least one enzyme selected from the group consisting of: papain, pancreatin, trypsin, chymotrypsin, pepsin, streptokinase, streptodornase, ficin, carboxypeptidase, aminopeptidase, chymopapain, bromelin, and subtilisin.

39. A cleansing composition for disinfecting articles that are susceptible to contamination by infectious prion protein, said composition comprising:
(i) one or more proteolytic protein(s) selected from the group consisting of keratinase enzymes, proteinase K, trypsins, chymotrypsins, pepsins, chymosins, cathepsins, subtilisins, elastases, collagenases, endopeptidases, peptidases, oligopeptidease, thermolysins, bacillolysin, mycilysins, carboxypeptidases, leucyl aminopeptidases, aminopeptidases, extremthermophilic proteases, carbonyl hydrolase, papain, pancreatin, streptokinase, streptodornase, ficin, carboxypeptidase, chymopapain, and bromelin; and
(ii) a solvent.

40. The cleansing composition of claim 39, comprising keratinase enzymes.

41. The cleansing composition of claim 40, wherein the concentration of said keratinase enzymes is with the range of from about 0.2 g/L to about 1.0 g/L.

42. The cleansing composition of claim 39, wherein the solvent is selected from the group consisting of distilled water, alcohol, buffer solution, and detergent solution.

43. The cleansing composition of claim 39, further comprising one or more chemical additives selected from the group consisting of surfactants, builders, boosters, and fillers.
